(19) United States
(12) Patent Application Publication
(10) Pub. No.: US 2026/0368490 A1
(43) Pub. Date: Nov. 19, 2026

(54) Advanced Machine Learning For Diagnostic and Prognostic Modeling of Stroke

(71) Applicants: REGENTS OF THE UNIVERSITY OF MICHIGAN, Ann Arbor, MI (US); Virginia Commonwealth University, Richmond, VA (US)

(72) Inventors: Emily Wittrup, Ann Arbor, MI (US); Aditya S. Pandey, Ann Arbor, MI (US); John Reavey-Cantwell, Richmond, VA (US); Kayvan Najarian, Northville, MI (US); Badih Junior Daou, Ann Arbor, MI (US); Dennis Rivet, Richmond, VA (US); Georgios Sioutas, Richmond, VA (US)

(21) Appl. No.: 19/675,944

(22) Filed: May 13, 2026

Related U.S. Application Data
(60) Provisional application No. 63/805,639, filed on May 14, 2025.

(30) Foreign Application Priority Data
May 13, 2025 (GR) 202500100362

(51) Int. Cl. G06N 3/0455; G16H 10/60

(52) U.S. Cl. CPC G06N 3/0455; G16H 10/60

(57) ABSTRACT

Methods and systems for generating a predicted outcome following a cerebrovascular accident are disclosed herein. A method includes: generating, via one or more processors of one or more autoencoders, at least one embedding vector for medical imaging data corresponding to the cerebrovascular accident, wherein at least one autoencoder of the one or more autoencoders is trained on historical medical images for a patient population associated with cerebrovascular accidents; generating, via one or more processors of the one or more autoencoders, contextual information for the medical imaging data based on historical electronic health record (EHR) data for the patient population; and generating, via one or more processors of a machine learning (ML) classifier model, a stroke outcome classification of the medical imaging data using the at least one embedding vector and the contextual information.

### CROSS-REFERENCE TO RELATED APPLICATIONS

This application claims the priority benefit under 35 U.S.C. § 119 of Greece Application No. 20250100362, filed May 13, 2025, and U.S. Provisional Application No. 63/805,639, filed May 14, 2025, which are both incorporated herein by reference in their entireties.

### FIELD OF THE DISCLOSURE

The present disclosure generally relates to using machine learning (ML) to predict outcomes following a cerebrovascular accident (stroke) and, more particularly, to techniques for evaluating medical imaging data for a patient using various machine learning models to generate outcome classification(s) for the medical imaging data.

### BACKGROUND

The background description provided herein is for the purpose of generally presenting the context of the disclosure. Work of the presently named inventors, to the extent it is described in this background section, as well as aspects of the description that may not otherwise qualify as prior art at the time of filing, are neither expressly nor impliedly admitted as prior art against the present disclosure.

Machine learning (ML) and artificial intelligence (AI) models are used by professionals in a wide variety of fields to analyze large quantities of data and make determinations based on said data. To properly analyze the data and/or make determinations, the ML and AI models often rely on rules programmed into the models in question. However, high performance models that are capable of analyzing complicated problems are often difficult for a user to understand. Notably, such “black box” models lack transparency and justification of the output recommendations and/or analysis, especially for the average individual user, who is often not trained in ML and/or AI programming.

Cerebrovascular accidents (strokes) are independently associated with significant morbidity and mortality. Additionally, duration and completion of recovery from a cerebrovascular accident may vary significantly between patients. Typically, medical imaging data (e.g., magnetic resonance imaging data, computed tomography imaging data, etc.), for a patient that has experienced a cerebrovascular accident, is evaluated by a clinician. However, such evaluations are highly variable between providers and centers, and may lead to erroneous diagnosis, classifications, treatment recommendations, etc. Due to the complexity of stroke management/intervention and the high stakes involved, present ML and AI models are not suitable for providing diagnosis, classifications, recommendations, etc., based on medical imaging data. Additionally, present ML and AI models are not equipped to provide interpretable models that allow clinicians to review and oversee the generation of classifications and recommendations.

### SUMMARY

In one embodiment, a computer-implemented method for generating a predicted outcome following a cerebrovascular accident may be provided. The computer-implemented method may be implemented via one or more local or remote processors, servers, sensors, transceivers, memory units, and/or other electronic or electrical components. The computer-implemented method may include: (1) generating, via one or more autoencoders, at least one embedding vector for medical imaging data corresponding to the cerebrovascular accident, wherein the at least one embedding vector is an output of an autoencoder trained on historical medical images for a patient population associated with cerebrovascular accidents; (2) generating, via one or more processors, contextual information for the medical imaging data based on historical electronic health record (EHR) data for the patient population; and (3) generating, via one or more processors of a machine learning (ML) classifier model, a stroke outcome classification of the medical imaging data using the at least one embedding vector and the contextual information.

In another embodiment, a system for generating a predicted outcome following a cerebrovascular accident may be provided. The system may include: one or more processors; one or more memories including computer-executable instructions that, when executed by the one or more processors, cause the computing system to: (1) generate, via one or more autoencoders, at least one embedding vector for medical imaging data corresponding to a cerebrovascular accident, wherein the at least one embedding vector is an output of an autoencoder trained on historical medical images for a patient population associated with cerebrovascular accidents; (2) generate contextual information for the medical imaging data based on historical electronic health record (EHR) data for the patient population; and (3) generate, via a machine learning (ML) classifier model, a stroke outcome classification of the medical imaging data using the at least one embedding vector and the contextual information.

In yet another embodiment, a tangible, non-transitory computer readable medium for generating a predicted outcome following a cerebrovascular accident may be provided. The tangible, non-transitory computer readable medium may include computer-executable instructions that, when executed by one or more processors of a computing system, cause the computing system to: (1) generate, via one or more autoencoders, at least one embedding vector for medical imaging data corresponding to a cerebrovascular accident, wherein the at least one embedding vector is an output of an autoencoder trained on historical medical images for a patient population associated with cerebrovascular accidents; (2) generate contextual information for the medical imaging data based on historical electronic health record (EHR) data for the patient population; and (3) generate, via a machine learning (ML) classifier model, a stroke outcome classification of the medical imaging data using the at least one embedding vector and the contextual information.

### BRIEF DESCRIPTION OF THE DRAWINGS

The accompanying figures, where like reference numerals refer to identical or functionally similar elements throughout the separate views, together with the detailed description below, are incorporated in and form part of the specification, and serve to further illustrate embodiments of concepts that include the claimed invention, and explain various principles and advantages of those embodiments.

FIG. 1 illustrates a block diagram of an example logic circuit for implementing example methods and/or operations for generating a predicted outcome following a cerebrovascular accident, in accordance with some embodiments described herein.

FIG. 2 illustrates a block-flow diagram depicting an example method for generating a predicted outcome following a cerebrovascular accident, implemented in the computing environment of FIG. 1, in accordance with some embodiments described herein.

FIG. 3 illustrates a block-flow diagram depicting an example architecture for an ensemble machine learning model, in accordance with some embodiments described herein.

FIG. 4 illustrates a block-flow diagram depicting an example architecture for an ensemble machine learning model, in accordance with some embodiments described herein.

FIG. 5 illustrates a graphical depiction of an example classification visualization, in accordance with some embodiments described herein.

FIG. 6 illustrates a graphical depiction of an example classification visualization, in accordance with some embodiments described herein.

FIG. 7 illustrates a graphical depiction of an example classification visualization, in accordance with some embodiments described herein.

FIG. 8 illustrates a flowchart depicting an example method for generating a predicted outcome following a cerebrovascular accident, implemented in the computing environment of FIG. 1, in accordance with some embodiments described herein.

Skilled artisans will appreciate that elements in the figures are illustrated for simplicity and clarity and have not necessarily been drawn to scale. For example, the dimensions of some of the elements in the figures may be exaggerated relative to other elements to help to improve understanding of embodiments of the present invention.

The apparatus and method components have been represented where appropriate by conventional symbols in the drawings, showing only those specific details that are pertinent to understanding the embodiments of the present invention so as not to obscure the disclosure with details that will be readily apparent to those of ordinary skill in the art having the benefit of the description herein.

### DETAILED DESCRIPTION

Generally, the techniques described herein relate to implementing machine-learning-based prediction systems that generate clinically relevant classifications, trajectories, and recommendations for patient outcomes associated with cerebrovascular accidents (strokes). More particularly, the disclosed techniques relate to utilizing medical imaging data (e.g., MRI, CT, or CTA data) and contextual information derived from electronic health records (EHR) to predict stroke outcomes and generate corresponding treatment recommendations.

Additionally, the disclosed techniques described herein provide further technical improvements by integrating embedding vectors derived from medical imaging data, generated using specialized embedding methods such as deep autoencoders. By reducing the dimensionality and extracting meaningful latent features from high-dimensional imaging data and EHR-derived clinical variables, the disclosed methods achieve improved predictive performance and computational efficiency (e.g., relative to generating outcome predictions directly from raw imaging and clinical datasets). In some embodiments, the invention described herein utilizes a learning with partially available privileged information (LUPAPI) framework that leverages complete medical datasets (e.g., including variables, or outcomes, not available for new patients at the time of prediction) enabling models to robustly generalize and remain operationally effective, even when patient information is incomplete or unavailable at decision-making time. These aspects combine to provide significant improvements in predictive modeling accuracy, computational efficiency, and clinical decision support capabilities not available using conventional outcome prediction methods.

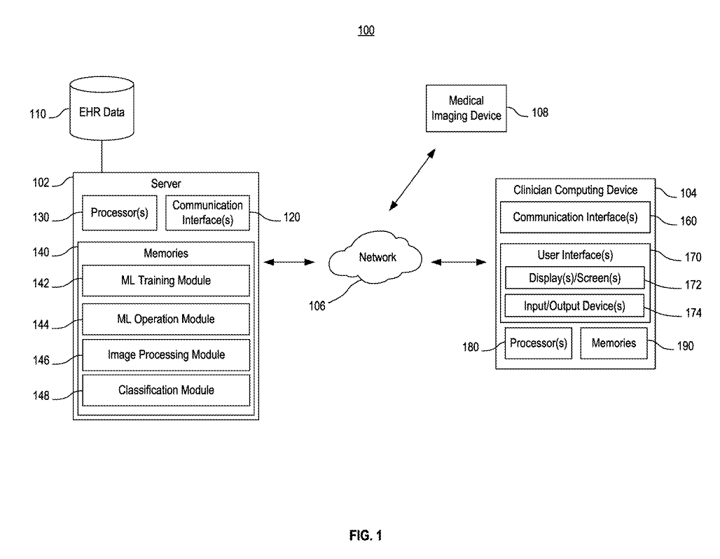

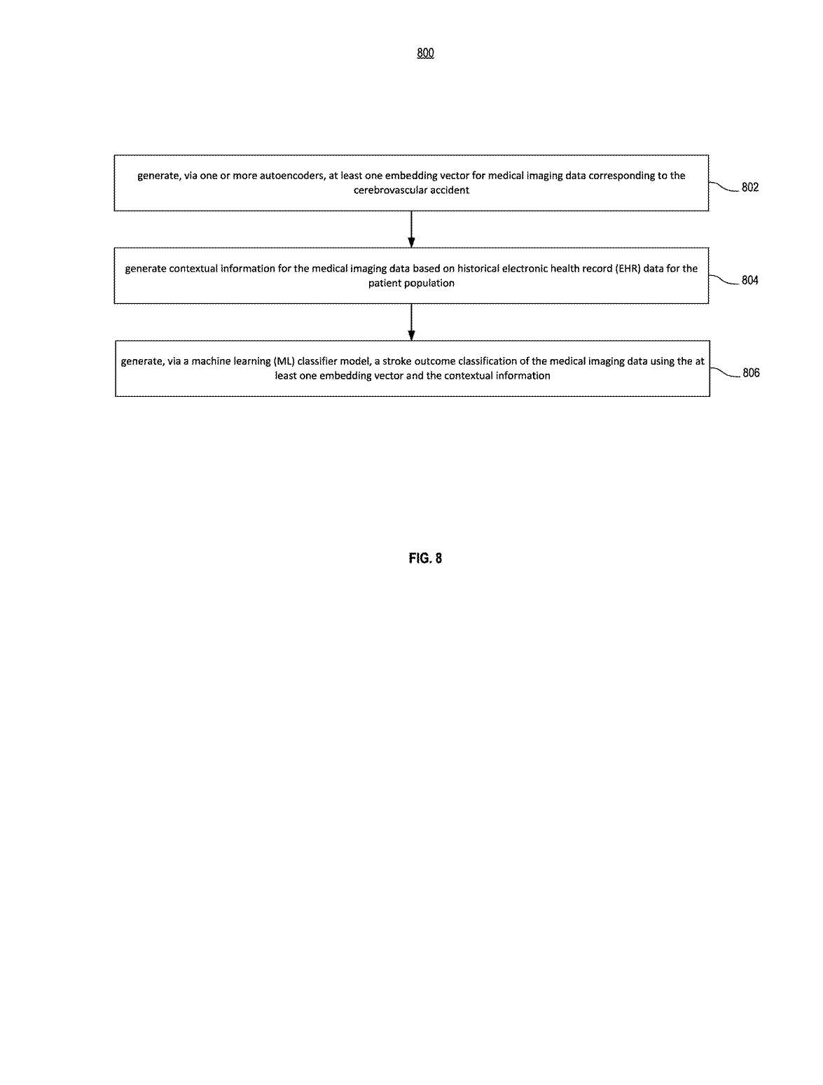

Referring now to the drawings, FIG. 1 is a block diagram representative of an example computing environment 100 capable of implementing the example methods and/or operations described herein, including, for example, one or more steps of the computer-implemented method 800 of FIG. 8 discussed in greater detail below. In some embodiments, the computing environment may be, or may be associated with a medical environment. The computing environment 100 of FIG. 1 includes a server computing device 102, a clinician computing device 104, and one or more networks 106. In some embodiments, the computing environment 100 further includes one or more medical imaging devices 108. Additionally or alternatively, the computing environment 100 may further include an electronic health record (EHR) database 110 and/or another database storing information for a patient population. The exemplary network 106 of FIG. 1 may be a single communication link directly connecting the server computing device 102 and the clinician computing device 104 (e.g., a direct wireless link), or one or more networks 106 may include multiple links (e.g., connecting the server computing device 102, the clinician computing device 104, the medical imaging device 108, the EHR database 110, and/or an additional computing device) and/or communication networks of one or more types (e.g., one or more wired and/or wireless local area networks (LANs), and/or one or more wired and/or wireless wide area networks (WANs) such as the Internet, public networks, private networks, etc.). The network 106 may include any number of network devices, such as gateways, switches, routers, modems, repeaters, and wireless access points, among others. Additionally or alternatively, the network 106 may also include computing devices such as computer servers. For ease of reading herein (and not for limitation purposes), the one or more networks 106 may be referred to using the singular tense.

The server computing device 102 may be an individual server, a group (e.g., cluster) of multiple servers, a computing device (e.g., a personal computer, a laptop, a smart phone, a tablet, a wearable device, etc.), or another suitable type of computing device or system (e.g., a collection of computing resources). The server computing device 102 may include one or more communication interfaces 120, one or more processors 130, and one or more memories 140. Further, the memories 140 may have stored thereon one or more modules (e.g., one or more sets of instructions) including a machine learning (ML) training module 142, a ML operation module 144, an image processing module 146, and a classification module 148.

The one or more communication interfaces 120 may enable communication with other machines (e.g., components of the computing environment 100 such as clinician computing device 104, medical imaging device 108, EHR database 110, etc.) via, for example, the one or more networks 106. The example communication interface 120 may include any suitable type of communication interface(s) (e.g., wired and/or wireless interfaces) configured to operate in accordance with any suitable protocol(s). For example, the communication interfaces 120 may be configured to transmit and receive data using a suitable wired communication protocol such as an Ethernet protocol, a USB protocol, a UART protocol, an I2C protocol, a SPI protocol, or wireless communication protocols such as a Bluetooth protocol, a Wi-Fi® (IEEE 802.11 standard) protocol, a near-field communication (NFC) protocol, a cellular (e.g., GSM, CDMA, LTE, WiMAX, etc.) protocol, a peer-to-peer wireless protocol, a short-range wireless protocol, and/or other suitable wired or wireless communication protocols. In some embodiments, the communication interface 120 may utilize a combination of such protocols to improve data throughput and/or efficiency. In some embodiments, the communication interface 120 may be a network interface controller (NIC) and may include any suitable NIC, such as wired/wireless controllers (e.g., Ethernet controllers), and facilitate bidirectional/multiplexed networking over the network 106 between the server computing device 102 and the clinician computing device 104 and/or other components of the environment 100 (e.g., medical imaging device 108, EHR database 110, etc.). In some embodiments, the communication interface 120 may include advanced features such as hardware acceleration, specialized networking protocols, etc.

The processors 130 may include one or more microprocessors, controllers, and/or any suitable type of processor, and the memories 140 (e.g., volatile memory, non-volatile memory, non-transitory computer-readable media, etc.) may be accessible by the processor 130 (e.g., via a memory controller). For example, the one or more processors 130 may include one or more central processing units, one or more graphics processing units, one or more field-programmable gate arrays, one or more application-specific integrated circuits, one or more tensor processing units, one or more digital signal processors, one or more neural processing units, one or more RISC-V processors, one or more coprocessors, one or more specialized processors/accelerators for artificial intelligence or machine learning-specific applications, one or more microcontrollers, etc. The processor 130 may interact with the memory 140 to obtain, for example, machine-readable instructions and/or computer-executable instructions stored in the memory 140 corresponding to, for example, the operations represented by the flowcharts of this disclosure (e.g., the computer-implemented method 800 of FIG. 8). For example, the memories 140 may include one or more random access memories, one or more read-only memories, one or more cache memories, one or more hard disk drives, one or more solid-state drives, one or more non-volatile memory express, one or more optical drives, one or more universal serial bus flash drives, one or more external hard drives, one or more network-attached storage devices, one or more cloud storage instances, one or more tape drives, etc. Additionally, the processors 130 may be communicatively coupled to and/or control the communication interface 120 to transmit and/or receive various information pursuant to executing of the instructions stored on the memories 140 via, for example, the network 106.

As noted above, the memories 140 may have stored thereon one or more modules, for example, as one or more sets of computer-executable instructions. Further, the memories 140 may include a ML training module 142, a ML operation module 144, an image processing module 146, and a classification module 148. In some aspects, the memories 140 may have stored thereon one or more additional modules (not depicted). For example, the memories 140 may include additional storage, such as one or more operating systems (e.g., Microsoft Windows, GNU/Linux, Mac OSX, etc.). The operating systems may be configured to run the modules stored on the memories 140 (e.g., modules 142, 144, 146, 148) during operation of the server computing device 102. The modules stored on the memories 140 may be implemented using any suitable computer programming language(s) (e.g., Python, JavaScript, C, C++, Rust, C#, Swift, Java, Go, LISP, Ruby, Fortran, etc.). Further, the modules stored on the memories 140 may be configured to communicate with one another (e.g., via inter-process communication, via a bus, via message queues, etc.). In some embodiments, the modules stored on the memories 140 may respond to network requests (e.g., via an application programming interface) or other requests received via the network 106 (e.g., via the user computing device 104 or other components of the environment 100). In some implementations, the memories 140 may store data structures and/or information related to, for example, the modules (e.g., modules 142, 144, 146, 148) and/or algorithms used in training, testing, or utilizing models to create, extract, and/or improve classification according to analyzed input data as described in more detail below.

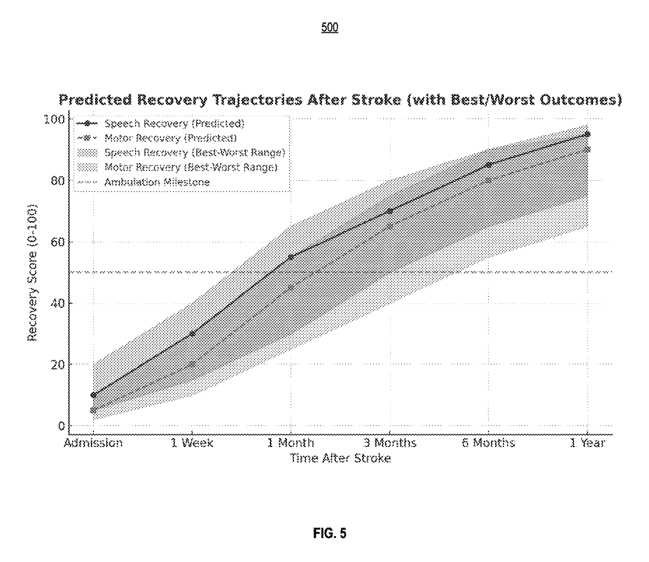

The server computing device 102 may also include one or more I/O devices (not depicted) and/or one or more displays/screens (not depicted), e.g., similar to the displays/screens 172 and the I/O devices 174 of the clinician computing device 104. Generally, the one or more I/O devices and/or the one or more displays/screens of the server computing device 102 may enable receipt of user input and communication of output data to the user. For example, the I/O devices and/or the displays/screens may present information in connection with the execution of instructions stored on the memories 140 for a user to view and/or otherwise perceive (e.g., the graphical user interfaces depicted in FIGS. 5,6 or 7). Similarly, the I/O devices and/or the displays/screens of the server computing device 102 may receive manual adjustments, selections, and/or data, and/or may allow a user to interact in any of a variety of manners with the processors 130 during execution of the instructions stored on the memories 140.

In some embodiments, a user may review data from, and/or provide data to, the modules of the memory 140 via the I/O devices of the server computing device 102 (not depicted) and/or the input/output (I/O) devices 174 of the clinician computing device 104. For example, the server computing device 102 may send data and/or information from modules 142, 144, 146, 148 to the computing device 104 to be presented via the user interface 170, and the server computing device 102 may receive inputs provided by a user (e.g., input data for modifying, adjusting, or otherwise interacting with a classification, model, etc.) via the user interface 170 from the computing device 104. In some embodiments, the server computing device 102 may send data and/or information from the modules 142, 144, 146, 148 to various components of the computing environment 100 (e.g., clinician computing devices 104, medical imaging devices 108, EHR database 110, and/or one or more additional computing devices). For example, the server computing device 102 may send data and/or information from the ML operation module 144 and/or the classification module 148 to one or more databases/datastores of the computing environment 100 (e.g., EHR database 110) for subsequent storage.

The clinician computing device 104 includes one or more communication interfaces 160, one or more user interfaces 170, one or more processors 180, and one or more memories 190. In some embodiments, the clinician computing device 104 may be included in and/or associated with a medical environment (e.g., similar to server computer device 102). The clinician computing device 104 may be a computing device (e.g., a personal computer, a laptop, a smart phone, a tablet, a wearable device, etc.), or another suitable type of computing device or system (e.g., a collection of computing resources). In some embodiments, one or more user computing devices 104 may be included in the computing environment 100 – for example, a user may access a first clinician computing device 104 that is a personal computer, a user may access a second clinician computing device 104 that is a smart phone, etc. Generally, a user of the clinician computing device 104 may be a clinician (e.g., a doctor, a physician, a surgeon, etc.) or another medical or healthcare professional (e.g., a nurse, a physician assistant, etc.). For ease of reading (and not limitation) purposes, the one or more user computing devices 104 may be referred to herein using the singular tense.

The one or more communication interfaces 160 may, similar to the communication interface 120, enable communication with other devices (e.g., server computing device 102) via, for example, the one or more networks 106. The example communication interface 160 may include any suitable type of communication interface(s) (e.g., wired and/or wireless interfaces such as a network interface controller) configured to operate in accordance with one or more suitable protocol(s). In some embodiments, the communication interface 160 may be a network interface controller (NIC) and may include any suitable NIC, such as wired/wireless controllers (e.g., Ethernet controllers), and facilitate bidirectional/multiplexed networking over the network 106 between the clinician computing device 104 and the server computing device 102 and/or other components of the environment 100 (e.g., medical imaging device 108, EHR database 110, another computing device, etc.). In some embodiments, the communication interface 160 may include advanced features such as hardware acceleration, specialized networking protocols, etc.

The one or more user interfaces 170 of the clinician computing device 104 may receive user input and communicate output data to the user. Further, the user interface 170 may be configured to present interactive graphical user interfaces (GUIs) to a user and receive feedback data from the user (e.g., via the input/output devices 174). For example, the user interface 170 may present interactive graphical user interfaces and/or graphical representations for a generated classification of a medical image, or imaging data, to a user (e.g., a clinician), such as the graphical user interfaces depicted in FIGS. 5, 6 or 7, as discussed in greater detail below. As another example, a clinician may provide feedback data, such as an adjusted classification, using the user interface 170. Continuing with this example, the input data from the user interface 170, from a clinician, may be sent to the server computing device 102 and the ML operation module 144 may update, change, modify, and/or retrain the model based on the feedback/input data.

The user interfaces 170 may include one or more input/output (I/O) device(s) 174. For instance, the I/O devices 174 may include one or more suitable types of user input devices, such as keyboards, touch screen displays, microphones, mice, touchpads, and/or any suitable types of remote and/or local user input devices. Similarly, the I/O devices 174 may include one or suitable types of output devices, such as touch screen displays, speakers, and the like. In some embodiments, the I/O devices 174 may include one or more display(s)/screen(s) 172 for presenting or displaying information to a user. The one or more displays/screens 172 may use any suitable display technology (e.g., LED, OLED, LCD, etc.). For example, the I/O devices 174 and/or the displays/screens 172 may be configured to present graphical representations of information and/or data associated with the embodiments described herein, such as information related to a trained model (e.g., a ML classifier, an autoencoder, etc.) or information related to training a model (e.g., information and/or data from the ML operation module 144). In some embodiments, the I/O device 174 and the display/screen 172 are integrated as a touchscreen display. In some embodiments, the user interfaces 170, the I/O devices 174, and/or the displays/screens 172 are not integral to the clinician computing device 104 and receive instructions from the clinician computing device 104 via wired and/or wireless transmissions over communication interface 160, for example. In some embodiments, the user interfaces 170, the I/O devices 174, and/or the displays/screens 172 may include one or more local interfaces, and/or may include one or more remote interfaces that are communicatively connected to the clinician computing device 104 via the network 106 (e.g., that are provided by an application, web browser, or other software executing on a device of a user). For ease of reading (and not limitation) purposes, the user interfaces 170, the I/O devices 174, and/or the displays/screens 172 may be referred to herein using the singular tense.

The processors 180 may include one or more microprocessors, controllers, and/or any suitable type of processor (e.g., one or more processors similar to the processors 130), and the memories 190 (e.g., one or more volatile memories, one or more non-volatile memories, or one or more non-transitory computer-readable media similar to the memories 140) may be accessible by the processor 180 (e.g., via a memory controller). The processor 180 may interact with the memory 190 to obtain, for example, machine-readable instructions and/or computer-executable instructions stored in the memory 190 corresponding to, for example, one or more of the operations represented by the flowcharts of this disclosure (e.g., the method 800 of FIG. 8). Additionally, the processors 180 may be communicatively coupled to and/or control the communication interface 160 to transmit and/or receive various information pursuant to executing of the instructions stored on the memories 190 and/or the memories 140 via, for example, the network 106.

As mentioned above, the memories 190 may have stored thereon one or more modules, for example, as one or more sets of computer-executable instructions. In some aspects, the modules may include additional storage, such as one or more operating systems (e.g., Microsoft Windows, GNU/Linux, Mac OSX, etc.), similar to, for example, memories 140. The operating systems may be configured to run the modules during operation of the clinician computing device 104. For example, the modules may include additional modules and/or services for receiving and processing data from one or more other components of the environment 100 such as the server computing device 102, the medical imaging device 108, or the EHR database 110. The modules may be implemented using any suitable computer programming language(s) (e.g., Python, JavaScript, C, C++, Rust, C#, Swift, Java, Go, LISP, Ruby, Fortran, etc.). The modules may be configured to communicate with one another (e.g., via inter-process communication, via a bus, via sockets, pipes, message queues, etc.). In some embodiments, the modules may respond to network requests or other requests received via the network 106 (e.g., via the server computing device 102 or other components of the environment 100).

The medical imaging devices 108 of the computing environment may be a medical imaging device capable and/or configured to capture imaging data of patients, or a particular body part of patients. For example, the medical imaging device 108 may be a magnetic resonance imaging (MRI) machine (e.g., ), a computed tomography (CT) machine (e.g., ), a computed tomography angiography (CTA) machine (e.g., ), or another type of medical imaging device (e.g., an X-ray machine). In some embodiments, the medical imaging device(s) 108 receives instructions from the server computing device 102 and/or the clinician computing device 104 via wired and/or wireless transmissions over communication interface 120 and/or communication interface 160 (e.g., and/or a communication interface of the medical imaging device 108), for example. Generally, the medical imaging device 108 may transmit medical imaging data (e.g., MRI data, CT imaging data, CTA imaging data, etc.) to the server computing device 102 and/or the clinician computing device 104. For ease of reading (and not limitation) purposes, the one or more medical imaging devices 108 may be referred to herein using the singular tense.

The electronic health record (EHR) database 110 of the computing environment 100 may store historical medical data associated with one or more populations, such as EHR data for one or more patient populations. For example, the patient population database 114 may store historical medical data associated with a population of patients who have experienced a cerebrovascular accident (a stroke). The historical medical data for a population stored on the EHR database 110 may include electronic health records (EHR) for a plurality of patients in the population and/or historical clinical data (e.g., procedural data, clinical guidelines, clinician notes, etc.) for a plurality of patients in the population. For example, the EHR data may include medical history data (e.g., past illnesses, past surgeries, family medical history, etc.), medication data, biographic information (e.g., gender, height, weight, age), medical conditions, etc. As another example, the historical clinical data may include vital signs for a patient over a period of time (e.g., vital signs data for the duration of a patient’s recovery from a surgery, other vital signs data collected for a patient over a period of time, routine check in/up data, and/or other clinical data collected over other periods of time). In some embodiments, the EHR database 110 may store data in one or more structured formats. For example, the EHR database 110 may store various sets of training data (e.g., within one or more vector data stores or caches) for the example machine learning models described herein.

Returning to the server computing device 102, the machine learning (ML) training module 142 stored on the memories 140 may include instructions for operating and/or training one or more machine learning models (e.g., an autoencoder, a machine learning classifier, etc.) and/or instructions for performing one or more of the operations described herein with respect to generating classifications of medical imaging data. In some embodiments, the ML operation module 144 and/or the classification module 148 may generate stroke outcome classifications for a patient based on medical imaging data associated with the patient using one or more example machine learning models (e.g., models trained via the ML training application 142). Typically, an example patient may be associated with one or more populations of patients (e.g., the patient has had a cerebrovascular accident, and the population consists of patients that have had cerebrovascular accidents). Further, the ML training module 142 may include instructions for training one or more machine learning models on EHR data and/or other medical information for a patient population associated with a patient of interest. In some embodiments, the ML training module 142 may train one or more ML models on clinical data such as medical information and documentation associated with the patient population (e.g., procedural data, clinical guidelines, clinician notes, medical interventions and associated outcomes, etc.). Additionally, the ML training module 142 may train one or more ML models on medical imaging data associated with a patient population. For example, the ML training module 142 may train one or more ML models on medical imaging data of a particular modality (e.g., MRI data, CT imaging data, etc.) and/or on medical imaging data of varying modality (e.g., a multi-modal ML model). In some embodiments, the ML training module 142 may train one or more ML models on medical imaging data, and/or corresponding embedding vectors, and contextual information associated with the medical imaging data (e.g., generated via one or more other trained ML models).

For example, the ML training module 142 may include instructions for training one or more autoencoders to generate embedding vectors for medical imaging data. Generally, an example autoencoder, trained via ML training module 142, may compress medical imaging data into lower dimensional embedding vectors. Furthermore, the ML training module 142 may an autoencoder on historical medical images associated with a particular patient population (e.g., a population associated with cerebrovascular accidents). Additionally or alternatively, the ML training module 142 may include instructions for training one or more autoencoders on historical EHR data for a patient population of interest to generate contextual information for medical imaging data.

As another example, the ML training module 142 may include instructions for training one or more ML classifier models on embedding vectors and contextual information, from one or more example autoencoders trained via ML training module 142, to generate classifications for the corresponding medical imaging data. In some embodiments, the ML training module 142 may generate an example ML classifier by training an artificial neural network (ANN) model to learn a function that maps a standard feature space of historical medical image embeddings and a privileged feature space of the historical EHR data to an outcome. Further, the ML training module 142 may train a model with information that is not available during an inference phase, sometimes referred to as learning with partially available privileged information (LUPAPI). For example, privileged information may include outcomes for historical patients associated with a particular population (e.g., the outcomes are known) and the ML training module 142 may provide such privileged information to a model during a training phase, whereas an outcome for a patient of interest associated with the population may be unknown (e.g., the patient is currently recovering, the outcome is ongoing/developing).

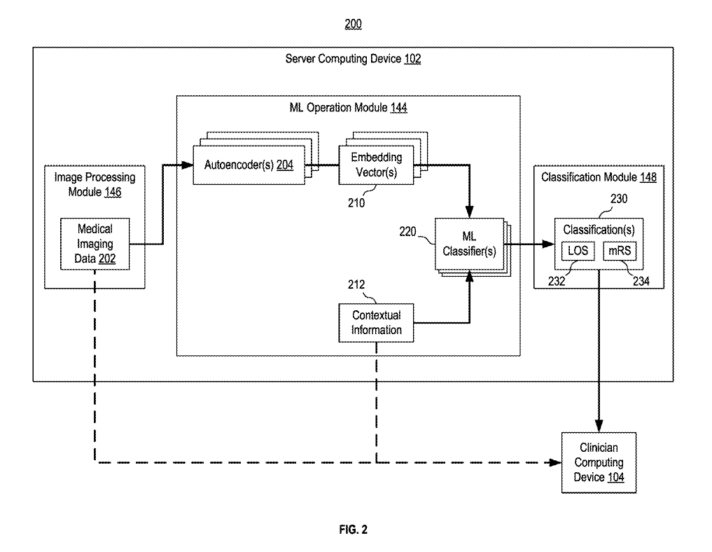

The ML operation module 144 stored on the memories 140 may include instructions for operating one or more example ML models described herein, such as one or more autoencoders, one or more ML classifiers, etc. Generally, the ML operation module 144 may include instructions for applying one or more ML models (e.g., trained via ML training module 142) to medical imaging data associated with a patient that has experienced a cerebrovascular accident to generate one or more stroke outcome classifications for the patient, as described further below with respect to FIGS. 2, 3 and 4.

For example, the ML operation module 144 may include instructions for generating one or more embedding vectors for medical imaging data associated with a cerebrovascular accident of a patient using one or more autoencoders. Further, the ML operation module 144 may apply an autoencoder, trained (e.g., via ML training module 142) on historical medical images for a patient population associated with a patient of interest, to medical imaging data for the patient of interest. In some embodiments, the ML operation module 144 receives/obtains multi-modal imaging data for a patient of interest (e.g., a patient that has had a cerebrovascular accident). Similar to the above, the ML operation module 144 may apply one or more autoencoders to medical imaging data for the patient of interest to generate one or more embedding vectors. For example, the ML operation module 144 may apply an individual multi-modal autoencoder (e.g., trained via ML training module 142 to process multi-modal imaging data) or multiple autoencoders (e.g., each trained via ML training module 142 to process a particular modality of imaging data) to such multi-modal imaging data.

As another example, the ML operation module 144 may include instructions for generating contextual information for medical imaging data associated with the cerebrovascular accident of the patient based on historical EHR data (e.g., from database 110) for the corresponding patient population using one or more autoencoders. Further, the ML operation module 144 may apply an autoencoder, trained (e.g., via ML training module 142) on historical EHR data for a corresponding patient population, to medical imaging data for a patient of interest. In some embodiments, the autoencoder may be further trained (e.g., via ML training module 142) on corresponding medical images for the patient population. Additionally or alternatively, the ML operation module 144 may apply an autoencoder (e.g., trained on historical EHR data and corresponding medical images) to medical imaging data and medical information (e.g., EHR data, clinician notes, vital signs data, active medical device data, etc.,) for a patient to generate the contextual information. In some embodiments, the contextual information may include relevant medical information identified (e.g., via one or more trained autoencoders) based on the medical imaging data for a patient, such as, procedural data, clinical guidelines, clinician notes, medical interventions and associated outcomes, etc.

As yet another example, the ML operation module 144 may include instructions for generating classifications of medical imaging data using one or more ML classifiers. Further, the ML operation module 144 may apply a ML classifier model, trained (e.g., via ML training module 142) on historical medical images and historical outcomes for patients associated with a particular population, to medical imaging data for a patient of interest and contextual information for the medical imaging data generated (e.g., via ML operation module 144) using one or more trained autoencoders. In some embodiments, the ML operation module 144 may generate classification for medical imaging data by applying one or more ML classifier models to the medical imaging data and EHR data for a patient. For example, the ML operation module 144 may obtain EHR data for the patient (e.g., from EHR database 110 or another medical information data source) and augment the contextual information with the EHR data for the patient.

The image processing module 146 stored on the memories 140 may include instructions for obtaining and processing medical imaging data from one or more components of the computing environment 100. For example, the image processing module 146 may obtain medical imaging data from the medical imaging device 108. In some embodiments, the image processing module 146 includes instructions for controlling the medical imaging device(s) 108 to capture medical imaging data and/or images for a patient. As another example, the image processing module 146 may obtain medical images from the EHR database 110, and/or another data store of the computing environment 100 storing historical medical images for a patient population. In some embodiments, the image processing module 146 may include instructions for processing medical imaging data (e.g., from EHR database 110, medical imaging device 108, another computing device). For example, the image processing module 146 may include instructions for mapping medical imaging data to a standardized coordinate system, such as the Montreal Neurological Institute (MNI) coordinate system. Additionally or alternatively, the image processing module 146 may include instructions for normalizing medical imaging data by applying a brain mask (e.g., a grey matter mask, a white matter mask, a whole brain mask, etc.) to the medical imaging data and/or images. The image processing module 146 may include instructions for processing, standardizing, and/or normalizing multi-modal imaging data and/or imaging data of varying modalities. For example, the image processing module 146 may process MRI data, CT imaging data, CTA imaging data, etc., for input to one or more ML models (e.g., via ML operation module 144).

The classification module 148 stored on the memories 140 may include instructions for generating stroke outcomes classifications for medical imaging data associated with a patient. For example, the classification module 148 may include instructions for generating a length of stay (LOS) classification, a recovery timeline classification (e.g.: recovery of motor functions, recovery of speech functions, predicted time to ambulation), a modified Rankin scale (mRS) classification (e.g., a classification measuring the degree of disability or dependence in daily activity of a patient), a discharge classification (e.g., discharge to home, discharge to inpatient rehabilitation, discharge to nursing facility, etc.), an intervention classification (e.g., need for a tracheostomy, need for a PEG tube, etc.), a mortality risk classification, and/or a combined or aggregated classification (e.g., incorporating features and/or concepts from the other classifications). The classification module 148 may also include instructions for presenting or displaying the example classification described herein. For example, the classification module 148 may present and/or display a classification and/or information associated therewith via the user interface 170. Expanding on this example, the classification module 148 may present classifications in a graphical form, as visual risk scores, as recovery trajectory timelines, as scenario-based simulations, as described further below with respect to FIGS. 5, 6 and 7. Additionally, the classification module 148 may present (e.g., via user interface 170) one or more stroke outcome classifications and the corresponding medical imaging data to facilitate review of the classification by a clinician or medical practitioner operating the user computing device 104. In some embodiments, the classification module 148 may provide the generated classification (e.g., from ML operation module 144) to one or more ML models, or generative AI models, to generate AI powered decision support information/context (e.g., a recommendation based on the generated classification, additional contextual information for the medical imaging data, etc.).

In some embodiments, the classification module 148 may generate various stroke outcome classification for a plurality of medical images associated with a patient and/or a cerebrovascular accident. As mentioned above, the ML operation module 144 may include instructions for obtaining EHR data associated with a patient to generate the stroke outcome classification for the corresponding medical imaging data. In some embodiments, the EHR data associated with the patient may include historical medical imaging data (e.g., historical MRIs, CT scans, etc.) for the patient of interest and the classification module 148 may generate (e.g., via ML operation module 144) one or more additional embedding vectors for the historical medical imaging data to be provided to the ML classifier model when tasked with generating a stroke outcome classification for the corresponding medical imaging data. Moreover, the classification module 148 may generate a stroke outcome classification for medical imaging data associated with a cerebrovascular accident of a patient based on historical medical images for the patient, thereby providing additional context to the ML classifier that may improve the accuracy and granularity of the generated stroke outcome classification.

In some embodiments, the medical imaging data obtained by the image processing module 146 (e.g., from the medical imaging device 108, from the EHR database 110, etc.) is multi-modal imaging data. Further, the classification module 148 may generate two or more embedding vectors for the multi-modal imaging data using at least two trained autoencoders (e.g., trained as described above with respect to the ML training module 142), with each vector of the two or more embedding vectors corresponding to a respective modality of the multi-modal imaging data. Additionally, the classification module 148 may generate a multi-modal stroke outcome classification of the medical imaging data via an ML classifier model (e.g., trained as described above with respect to the ML training module 142) and using the two or more embedding vectors and the contextual information for the medical imaging data.

In some embodiments, the medical imaging data obtained by the image processing module 146 includes a time series of medical images (e.g., medical images for a patient captured over a period of time, such as hours, days, weeks, months, etc.). Further, the classification module 148 may generate, via one or more trained autoencoders, one or more additional embedding vectors (e.g., in addition to the an embedding vector for the initial, or most recent, medical image or imaging data) for one or more additional medical images in the time series of medical images, with each additional embedding vector corresponding to a respective medical image of the time series of medical images. Additionally, the classification module 148 may generating a time-series stroke outcome classification of the medical imaging data using the additional embedding vectors, and the embedding vector for the initial medical imaging data and the corresponding contextual information.

In some embodiments, the classification module 148 may include instructions for generating, via a trained ML model (e.g., a trained ML classifier), a treatment recommendation for medical imaging data associated with a patient of interest and/or a cerebrovascular accident for the patient. For example, the classification module 148 may generate, via a trained ML model, a treatment recommendation based on the at least one embedding vector for the medical imaging data, the contextual information for the medical imaging data, and/or the stroke outcome classification for the medical imaging data.

In some embodiments, the image processing module 146 may receive and process volumetric (3D) medical imaging data or datasets. Further, conventional image-based clinical decision support systems rely predominantly on specific 2D slices or limited anatomical cross-sections, potentially missing clinically relevant spatial relationships or broader anatomical context within the complete 3D volume. In contrast, the techniques described herein include utilizing trained autoencoders to efficiently process entire volumetric images, thereby capturing richer and more comprehensive latent feature representations of complex brain anatomy, for example. Further, the ML training module 142 may train an autoencoder on historical volumetric imaging data to generate embedding vectors that capture clinically meaningful patterns across the entire volumetric context. Using the conventional techniques, employing comprehensive volumetric imaging data in this manner requires relatively large and computationally intensive model architectures, making training more resource-demanding as compared to conventional 2D approaches. However, the modular nature of the example autoencoders described herein substantially counteracts these challenges by enabling a single initial training step. Specifically, an example autoencoder (e.g., autoencoders, 304, 306) may be trained once on representative historical volumetric imaging data (e.g., volumetric imaging data representative of a patient population) to generate embedding vectors that can be reused multiple times across diverse downstream prediction tasks without requiring additional re-training. Thus, the disclosed embodiments may, while leveraging complete volumetric imaging information, reduce overall computational complexity and improve predictive performance.

In some embodiments, modules 142, 144, 146, 148 may include additional computer-executable instructions corresponding to, for example, one or more of the operations represented by the flowcharts of this disclosure (e.g., the computer-implemented method 800 of FIG. 8). For example, the ML operation module 144 and/or the classification module 148 may include instructions for generating a stroke outcome classification for medical imaging data corresponding to a cerebrovascular accident associated with a patient. Further, the modules stored on the memories 140 may include instructions for operating and applying (e.g., to medical imaging data and associated contextual information) various trained machine learning models, as discussed further below with respect to FIGS. 2-8.

Referring next to FIG. 2, a block-flow diagram illustrates a method 200 for generating a predicted outcome following a cerebrovascular accident. The method 200 of FIG. 2 may be implemented by the processors 130, the processors 180, and/or other suitable processors, etc., executing instructions stored on the memories 140, the memories 190, and/or another suitable non-transitory computer readable medium, etc., described above with respect to FIG. 1. Though the method below is described with regard to the computing environment 100, it will be recognized that any similarly suitable environment(s) and/or system(s) may be used to implement method 200.

As described above, the memories 140 of the server computing device 102 may include one or more modules such as an ML training module 142, an ML operation module 144, an image processing module 146, and a classification module 148. Generally, the method 200 may include obtaining or receiving medical imaging data (block 202) at the image processing module 146, as described above with respect to FIG. 1 (e.g., the medical imaging data may be provided to or obtained from a component of the computing environment 100, such as the medical imaging device 108 or the EHR database 110). For example, the medical imaging data 202 may correspond to a cerebrovascular accident associated with a patient. The image processing module 146 may apply preprocessing operations to the medical imaging data 202, such as mapping the medical imaging data to a standardized coordinate system and normalizing the medical imaging data using a brain mask (e.g., as described above with respect to FIG. 1). The method 200 may further include providing the preprocessed imaging data 202 to the ML operation module 144.

The ML operation module 144 may provide the medical imaging data 202 to one or more autoencoders (block 204). For example, an autoencoder 204 may be a machine learning model, such as an autoencoder, trained (e.g., via ML training module 142 as described above with respect to FIG. 1) on historical medical images of a particular modality, anatomical region, demographic of patient, etc., to generate embedding vectors and reduce the dimensionality of input feature vectors. In some embodiments, an example autoencoder may be a ML model trained (e.g., via ML training module 142 as described above with respect to FIG. 1) on historical clinical/medical information such as electronic health record (EHR) data, procedural data, clinical guidelines, clinician notes, medical interventions and associated outcomes, etc., and historical medical images associated with a patient population including the patient, to generate contextual information (e.g., relevant clinical information) associated with input medical imaging data.

Furthermore, the method 200 may include generating one or more embedding vectors (block 210) using the one or more autoencoders 204. For example, the medical imaging data 202 may include multi-modal imaging data (e.g., MRI data and CTA imaging data) and the ML operation module 144 may provide the medical imaging data 202, or a portion of the imaging data, to a first feature extraction model trained for a first modality (e.g., MRI data), a second feature extraction model trained for a second modality (e.g., CTA imaging data), etc.

Additionally, the method 200 may include generating contextual information (block 212) using an autoencoder. For example, an additional autoencoder (not depicted) may be trained (e.g., via ML training module 142) on historical medical images and EHR data for the corresponding patient population and an additional autoencoder (e.g., one of the autoencoders 204) may be trained (e.g., via ML training module 142) on historical medical images and other clinical data, such as procedural data and clinical guidelines.

The method 200 may include providing the embedding vector(s) 210 and the contextual information 212 to one or more ML classifier models (block 220). As described above, the ML training module 142 may train one or more ML classifier models to generate stroke outcome classifications for medical imaging data. Further, the ML operation module 144 may generate one or more classifications (block 230) for the medical imaging data 202 by inputting the one or more embedding vectors 210 and the contextual information 212 to the one or more ML classifier models 220. For example, a first ML classifier of the one or more ML classifiers 220 may be trained to generate a length of stay (LOS) classification (block 232) for input embedding vectors and a second ML classifier may be trained to generate a modified Rankin scale (mRS) classification for input embedding vectors. As described above with respect to FIG. 1, an example ML classifier may be trained to generate a LOS classification (e.g., predicted timelines for: recovery of motor functions, recovery of speech functions, predicted time to ambulation), a mRS classification (e.g., a classification measuring the degree of disability or dependence in daily activity of a patient), a discharge classification (e.g., discharge to home, discharge to inpatient rehabilitation, discharge to nursing facility, etc.), an intervention classification (e.g., need for a tracheostomy, need for a PEG tube, etc.), and/or a combined or aggregated classification (e.g., incorporating features and/or concepts from the other classifications). In would be understood that the method 200 may include generating any number of such classifications for the medical imaging data 202.

The classification module 148 may obtain or receive the classifications 230 from the ML classifier 220 including, for example, a LOS classification 232 and a mRS classification 234. The method 200 may include providing the classification 230, 232, and 234 to the clinician computing device 104 (e.g., via the network 106). For example, the classification module 148 may generate one or more graphical representation and/or other visualizations of the generated classifications, as described below with respect to FIGS. 5-7. In some embodiments, the method 200 includes providing the medical imaging data 202 and the contextual information 212 to the clinician computing device 104 (e.g., to be reviewed by a clinician) with, for example, the classifications 230, 232, 234.

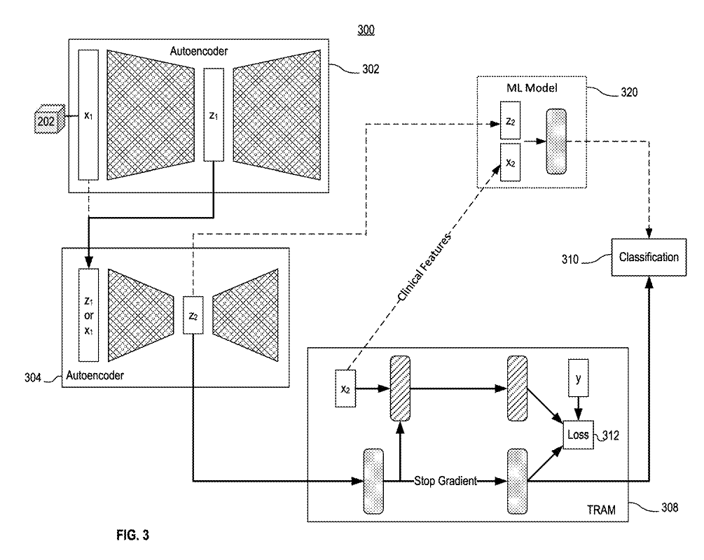

Referring next to FIG. 3, a block-flow diagram illustrates an architecture 300 for an ensemble machine learning model. The model architecture 300 of FIG. 3 may be implemented by the processors 130, the processors 180, and/or other suitable processors, etc., executing instructions stored on the memories 140, the memories 190, and/or another suitable non-transitory computer readable medium, etc., described above with respect to FIG. 1. Though the method below is described with regard to the computing environment 100, it will be recognized that any similarly suitable environment(s) and/or system(s) may be used to implement the model architecture 300.

Generally, the architecture 300 may process medical imaging data through a multi-step encoding, feature extraction, augmentation, and classification pipeline. Initially, medical imaging data 202, or x₁, may be provided to a first autoencoder 302. The autoencoder 302 employs an encoding-decoding structure (e.g., encoding the initial input data x₁ into a lower-dimensional latent representation z₁). The latent representation z₁ may encapsulate underlying structural and contextual features of the medical imaging data 202, thereby facilitating more robust downstream tasks such as clinical feature extraction and classification.

The latent features z₁ generated by the first autoencoder 302 may be provided as input to a second autoencoder 304. Additionally or alternatively, the initial input data x₁ may be provided as input to the second autoencoder 304. Similar to autoencoder 302, the second autoencoder 304 may employ an encoding-decoding mechanism to further refine or extract latent features z₂ (e.g., deeper, more context-rich latent features, relative to latent features z₁). Furthermore, the selection of x₁ and/or z₁ as the input to autoencoder 304 supports multiple strategies for representation learning, allowing optimized tuning depending on the intended clinical scenario or classification task.

The refined latent features z₂ generated by the second autoencoder 304 may be provided as input to a transfer and marginalized (TRAM) network 308. Additionally, clinical data x₂, or clinical features, may be provided to the transfer and marginalized (TRAM) network 308. For example, the clinical data x₂ may correspond to clinical target labels y (e.g., clinical outcomes), such as clinically derived ground truth information.

Generally, the transfer and marginalized (TRAM) network 308 of FIG. 3 may function as a specialized machine learning component configured to integrate and analyze both latent feature representations (e.g., z₂ generated by autoencoder 304) and additional clinical feature inputs or clinical data (e.g., x₂). Specifically, the TRAM network 308 may synthesize latent image-derived features z₂ (e.g., embedding vectors 210 of FIG. 2) along with auxiliary clinical features x₂ (such as clinician notes, EHR data, demographic patient information, alternate imaging modalities, patient history, procedural information, or combinations thereof) to generate a classification 310 for the medical imaging data 202. By analyzing both latent image features and clinically relevant data points, the TRAM network 308 may leverage multimodal data to generate classification predictions closely aligned with clinical outcomes. Generally, the classification 310 may include clinically relevant outcomes such as cerebrovascular accident prognosis, length of stay (LOS) classifications, modified Rankin scale (mRS) classification, intervention recommendations, discharge planning decisions, or similar diagnostic or prognostic predictions.

In some embodiments, the TRAM network 308 leverages a strategy referred to as learning with partially available privileged information (LUPAPI). Generally, with LUPAPI, the TRAM network 308 may employ privileged clinical information which may be fully accessible during model training (e.g., historical patient EHR data, clinician notes, chronic medical conditions, or additional clinical contextual variables), but only partially or not available at all during inference (e.g., clinical information for a particular patient is missing), when the model processes new patient data. In this manner, the TRAM network 308 utilizes fully available privileged knowledge at training time, enabling the encoder and classifier components to learn highly robust and clinically relevant latent and clinical feature representations. As a result, even if some privileged clinical details are incomplete or entirely absent in operational scenarios, such as during clinical decision-making for new stroke cases, the TRAM network 308 effectively generalizes to provide reliable predictions based on the available latent imaging-derived features (z₂) and clinical features (x₂) alone.

Furthermore, by employing LUPAPI within the TRAM network 308, classification outcomes may be clinically informed by rich historical clinical data sets and advanced medical knowledge accumulated over large patient populations. For example, historical EHR data representing privileged clinical information may be integrated together with latent embedding vectors from historical medical images during the network training phase. This enables advanced artificial neural networks within the ML model 320 or TRAM network 308 structure to accurately learn intricate mappings between multimodal historical patient data and clinical outcomes. Consequently, even when certain clinical variables or privileged information are only partially available for new patients, predictions remain highly accurate and clinically meaningful, ultimately supporting efficient and reliable clinical interventions, timely patient resource allocation, as well as improved healthcare outcomes.

Additionally, the TRAM network 308 may include a stop gradient for selectively controlling gradient flow during training, strategically isolating certain upstream or parallel network components (such as autoencoders 302, 304 or other embedding pathways) from direct backpropagation of loss information. By selectively controlling gradient propagation, the TRAM network 308 may encourage stable and targeted updates to its internal parameters, enhancing the quality and clinical relevance of the generated classifications/predictions.

As illustrated in FIG. 3, the TRAM network 308 employs a loss computation step 312, measuring the discrepancy between generated classification/predictions and clinically defined target labels y, or ground truth outcomes (e.g., known clinical outcomes, expert-determined prognoses, or other derived clinical labels). Generally, the loss computed by the TRAM network 308 (block 312) may be used to refine the internal parameters of TRAM network 308, thereby improving the generated classifications 310 derived from the latent features z₂ and clinical feature x₂.

In some embodiments, a ML model 320 generates classification 310 based on latent features z₂ from autoencoder 304 and clinical features x₂. As discussed above, clinical features (e.g., contextual information 212) processed by the ML model 320 may represent underlying information such as relevant imaging-derived features (e.g., from historical imaging data), patient clinical history, electronic health record (EHR) data, associated medical modalities, or other clinically significant inputs that support robust predictive analysis. Similar to TRAM network 308, by analyzing and integrating latent embedding vectors z₂ and clinical data x₂, the ML model 320 forms robust clinical representations to generate classifications 310.

In summary, the ensemble machine learning architecture 300 may be configured to generate clinically relevant predictive outputs (e.g., classification 310) using hierarchical encoding (e.g., autoencoders 302, 304), clinical data integration (e.g., x₂), representation learning with selective gradient control (e.g., TRAM network 308), and/or robust clinical feature extraction (ML model 320). Furthermore, the example architecture 300 supports comprehensive medical imaging analysis, enhancing clinical interpretability, predictive accuracy, and diagnostic or prognostic decision-making processes.

Additionally, the ensemble machine learning architecture 400 can leverage parallel deep-feature extraction (via autoencoders 302, 304) and clinical-context generation (via TRAM network 308) to analyze complex medical imaging data and clinical context in a coordinated fashion. By integrating multimodal embedding representations (both image-derived and contextually-informed) along with relevant external data sources (EHR data, imaging-derived features, historical patient data), the ML model 320 may accurately generate clinically actionable predictions for stroke-related patient outcomes (e.g., LOS, mRS, intervention requirement, or discharge decisions).

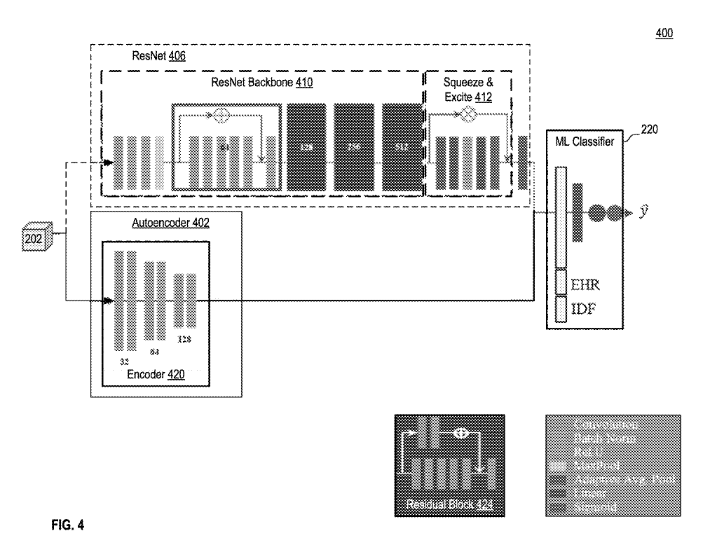

Referring next to FIG. 4, a block-flow diagram illustrates an architecture 400 for an ensemble machine learning model. The model architecture 400 of FIG. 4 may be implemented by the processors 130, the processors 180, and/or other suitable processors, etc., executing instructions stored on the memories 140, the memories 190, and/or another suitable non-transitory computer readable medium, etc., described above with respect to FIG. 1. Though the method below is described with regard to the computing environment 100, it will be recognized that any similarly suitable environment(s) and/or system(s) may be used to implement the model architecture 400.

As depicted in FIG. 4, the medical imaging data 202 may be provided (e.g., via image processing module 146, as described above with respect to FIGS. 1-2) to the ensemble machine learning architecture 400. In some embodiments, the architecture 400 is a simplified version of the architecture 300 of FIG. 3. As shown in FIG. 4, the architecture 400 includes an autoencoder 402 (e.g., a autoencoder 204, autoencoder 302, autoencoder 304) consisting of an encoder 420. Generally, the autoencoder 402 may be trained to compress high dimensional medical imaging data into lower-dimensional, clinically meaningful feature representations, or embedding vectors.

Generally, the encoder 420 of the autoencoder 402 may include convolutional layers and ReLU layers arranged in structured channel depths (e.g., 32, 64, and 128). In comparison to conventional approaches, the autoencoder 402 provides improved computational efficiency and generates embedding vectors with richer and more clinically relevant latent features. Further the autoencoder 402 is specifically trained on historical imaging data, and in some cases associated clinical context, to generate embedding representations that more effectively capture clinically meaningful feature patterns contained in high-dimensional medical imaging inputs. Additionally, the autoencoder-based approach described herein provides significant operational advantages over general CNN classifier architectures such as residual networks. For instance, due to their modular nature, an example autoencoder requires only a single, initial training; in contrast, conventional feature extraction models typically necessitate retraining from scratch for each prognostic outcome of interest, resulting in substantially increased computational overhead. Further, the modular and flexible structure of autoencoder 402 lends itself to implementations leveraging advanced frameworks, such as learning using partially available privileged information (LUPAPI), enabling more robust handling of incomplete patient data.

Subsequently, the outputs(s) from autoencoder 402 (i.e., extracted imaging features / embedding vectors) are provided to a machine learning (ML) classifier model 220. As discussed above, the ML classifier 220 integrates clinically relevant inputs, such as imaging-derived features (IDF) and electronic health record (EHR) data (e.g., obtained from EHR database 110) to generate classifications for medical imaging data. After integrating these multimodal inputs—consisting of the imaging-derived embedding vectors from autoencoder 402 and supplementary data such as EHR data and IDF data (e.g., training data provided to the ML classifier 220, additional context provided to the ML classifier, etc.)—the ML classifier 220 may generate a classification (ŷ), for example, predicting stroke outcomes, length of stay (LOS), modified Rankin scale (mRS) scores, or other clinical classifications as described with respect to FIGS. 1-2.

In some embodiments, the architecture 400 may employ alternative neural network architectures, such as conventional deep learning backbones or other lightweight networks, to extract features from medical imaging data. For example, the architecture 400 may include a Residual Network (ResNet) 406 consisting of a ResNet Backbone 410 and squeeze and excite layers 412, as shown in FIG. 4. While medical image feature extraction is primarily discussed with respect to autoencoders, and such deep learning alternatives are primarily discussed for comparative context, it would be understood that alternative architectures such as ResNet 406 (and/or other conventional deep learning architectures, backbones, etc.) simply represent optional alternatives.

The ResNet 406 may include a ResNet backbone 410, such as a ResNet 10 backbone or another similar lightweight network. The ResNet backbone 410 may include several sequential operations, such as convolution layers, batch normalization (Batch Norm) layers, Rectified Linear Unit (ReLU) activation layers, and max pooling (MaxPool) layers. For example, the ResNet backbone 410 may include one or more residual blocks, such as residual block 424, comprising additional convolutional layers, batch normalization layers, and/or ReLU layers that employ residual connections to enhance feature representation and layer stability. In the illustrated example, the ResNet backbone 410 includes feature map depth transitions (e.g., changes in the number of filters) denoted by indicated numerical values (e.g., 64, 128, 256, and 512). Additionally, the ResNet backbone 410 may include residual connections to facilitate efficient training and improved learning by mitigating gradient vanishing problems inherent to deep neural architectures.

In addition to the ResNet backbone 410, the ResNet 406 may further include squeeze & excite (SE) layers 412. The SE layers 412 may include adaptive average pooling layers, sigmoid layers, linear layers, and ReLU layers. For example, the SE layers 412 may utilize channel-wise attention mechanisms to recalibrate intermediate feature maps and enhance discriminative feature representation. Further, the SE layers 412 leverage channel-wise dependencies to emphasize informative features.

Referring next to FIG. 5, a graphical depiction illustrates a classification visualization 500 for a predicted outcome following a cerebrovascular accident. The visualization 500 of FIG. 5 may be presented via a user interface, such as the user interface 170 of the clinician computing device 104, in response to the processors 130, the processors 180, and/or other suitable processors, etc., executing instructions stored on the memories 140, the memories 190, and/or another suitable non-transitory computer readable medium, etc., described above with respect to FIG. 1. Though the visualization below is described with regard to the computing environment 100, it will be recognized that any similarly suitable environment(s) and/or system(s) may be used to present the visualization 500.

Specifically, the visualization 500 includes a graph showing predicted recovery trajectories after stroke (with best/worst outcomes), with the x-axis representing time after stroke, beginning at an initial time point (e.g., at admission) and progressing through subsequent intervals (e.g., a week, a month, three months, a year, etc.), and the y-axis representing a recovery score (e.g., ranging from 0 to 100) indicative of the level of patient recovery achieved over the indicated time span. For example, a length of stay (LOS) classification, generated as described above with respect to FIG. 1-4, may include recovery scores over various time spans. The visualization 500 may include predictive trajectories for both speech function recovery and motor function recovery, and corresponding trajectory ranges (e.g., best-worst outcome ranges). Lastly, the visualization 500 may include an ambulation milestone, the horizontal line at approximately a recovery score of 50, signifying a critical clinical threshold generally associated with independent ambulation or significant mobility improvement.

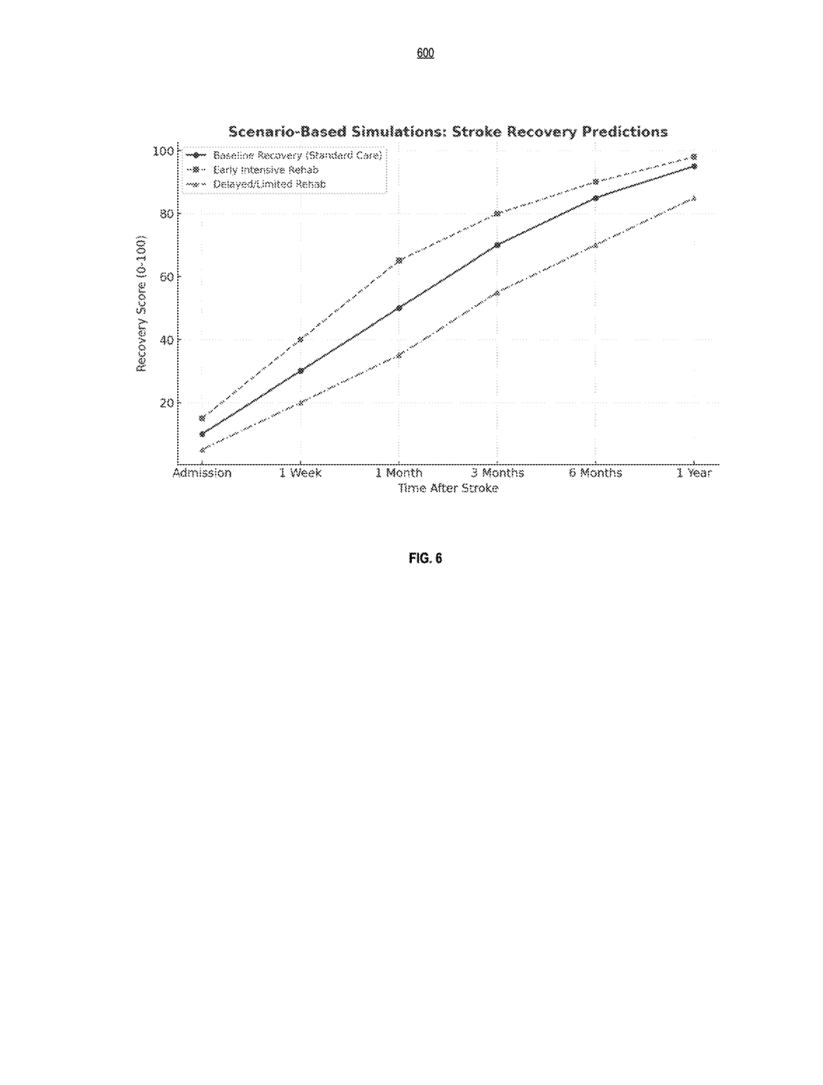

Referring next to FIG. 6, a graphical depiction illustrates a classification visualization 600 for a predicted outcome following a cerebrovascular accident. The visualization 600 of FIG. 6 may be presented on a user interface, such as the user interface 170 of the clinician computing device 104, in response to the processors 130, the processors 180, and/or other suitable processors, etc., executing instructions stored on the memories 140, the memories 190, and/or another suitable non-transitory computer readable medium, etc., described above with respect to FIG. 1. Though the visualization below is described with regard to the computing environment 100, it will be recognized that any similarly suitable environment(s) and/or system(s) may be used to present the visualization 600.

Specifically, the visualization 600 includes a graph illustrating scenario-based simulations of predicted stroke recovery trajectories for various rehabilitation strategies. The x-axis represents time after stroke and the y-axis represents a recovery score (e.g., ranging from 0 to 100) indicative of the level of patient recovery achieved with the particular rehabilitation strategy. The visualization 600 may include predicted trajectories corresponding to: (1) baseline recovery under standard care, (2) recovery under early intensive rehabilitation, and (3) recovery under delayed or limited rehabilitation.

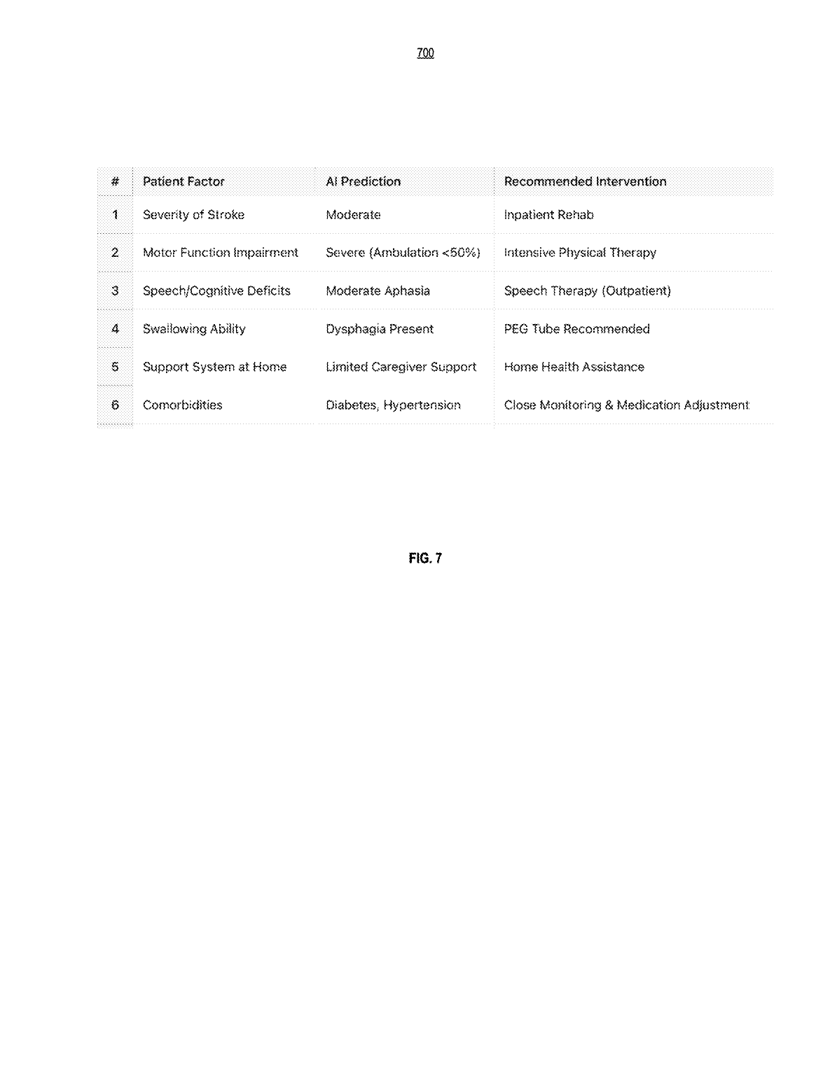

Referring next to FIG. 7, a graphical depiction illustrates a classification visualization 700 for a predicted outcome following a cerebrovascular accident. The visualization 700 of FIG. 7 may be presented on a user interface, such as the user interface 170 of the clinician computing device 104, in response to the processors 130, the processors 180, and/or other suitable processors, etc., executing instructions stored on the memories 140, the memories 190, and/or another suitable non-transitory computer readable medium, etc., described above with respect to FIG. 1. Though the visualization below is described with regard to the computing environment 100, it will be recognized that any similarly suitable environment(s) and/or system(s) may be used to present the visualization 700.

Specifically, the visualization 700 includes a table visualization predicted outcomes and/or classification and recommendations for a patient (e.g., generated based on medical imaging data for the patient as described above with respect to FIG. 1-6). For example, the visualization 700 may include columns for patient factors or clinical categories, generated predictions, and/or recommended interventions. Generally, each of row of the visualization 700 may correspond to a specific clinical patient factor. For example, stroke outcome classifications and corresponding treatment recommendations, generated as described above with respect to FIG. 1-6, may be presented in the AI prediction and recommended intervention columns, respectively.

Referring next to FIG. 8, a flowchart illustrates a method 800 for generating a predicted outcome following a cerebrovascular accident. The method 800 of FIG. 8 may be implemented by the processors 130, the processors 180, and/or other suitable processors, etc., executing instructions stored on the memories 140, the memories 190, and/or another suitable non-transitory computer readable medium, etc., described above with respect to FIG. 1. Though the method below is described with regard to the computing environment 100, it will be recognized that any similarly suitable environment(s) and/or system(s) may be used to implement method 800.

At block 802, a computing device (e.g., server computing device 102) may generate at least one embedding vector for medical imaging data corresponding to the cerebrovascular accident (e.g., a cerebrovascular accident associated with a patient of interest). In some embodiments, the medical imaging data is magnetic resonance imaging (MRI) data, computed tomography (CT) imaging data, or computed tomography angiography (CTA) imaging data. For example, the ML operation module 144 may generate, via one or more autoencoders (e.g., autoencoders 204), at least one embedding vector for input medical imaging data. Further, at least one autoencoder of the one or more autoencoders may be trained (e.g., via ML training module 142) on historical medical images for a patient population associated with cerebrovascular accidents. For example, the at least one embedding vector for an input medical image may be an output of an autoencoder trained on historical medical images from EHR database 110 or another patient information data store of the computing environment 100. In some embodiments, the method 800 may further include training, via one or more processors, the at least one autoencoder on a historical medical imaging dataset to reduce dimensionality of input feature vectors. For example, imaging data included in the historical medical imaging dataset may be mapped to a standardized coordinate system and normalized by applying a brain mask. In some embodiments, the at least one autoencoder of the one or more autoencoders includes a squeeze and excitation network.

At block 804, the computing device may generate contextual information for the medical imaging data based on historical electronic health record (EHR) data for the patient population (e.g., EHR data from EHR database 110). For example, the ML operation module 144 may generate (e.g., via TRAM network 408, a trained autoencoder) contextual information for input medical imaging data.

At block 806, the computing device may generate a stroke outcome classification of the medical imaging data using the at least one embedding vector and the contextual information. For example, the ML operation module 144 may generate, via a machine learning (ML) classifier (e.g., ML classifier 220, ML classifier 320, TRAM network 308), one or more stroke outcome classifications for input medical imaging data (e.g., based on corresponding embedding vector(s) and/or contextual information generated via the autoencoders described herein). In some embodiments, the historical EHR data includes partially available privileged information for the patient population, and the method 800 further includes training, via one or more processors of the ML classifier model, an artificial neural network model to learn a function that maps a standard feature space of historical medical image embeddings and a privileged feature space of the historical EHR data to an outcome. In some embodiments, the stroke outcome classification includes a length of stay (LOS) classification, a modified Rankin scale (mRS) classification, a recovery timeline classification, or an intervention classification. In some embodiments, at least one of the one or more autoencoders may be trained on historical electronic health record (EHR) data for the patient population.

Embodiments of the method 800 may further include obtaining, via one or more processors, EHR data associated with the medical imaging data. For example, the stroke outcome classification may be generated using the EHR data associated with the medical imaging data. In some embodiments, the EHR data associated with the medical imaging data includes additional historical medical imaging data. Furthermore, the method 800 may include generating, via the one or more autoencoders, at least one additional embedding vector for the additional historical medical imaging data. For example, the stroke outcome classification may be generated using the at least one additional embedding vector.

In some embodiments, the medical imaging data is multi-modal imaging data, and the method 800 further includes generating, via the one or more autoencoders, two or more embedding vectors for the medical imaging data and each embedding vector of the two or more embedding vectors may correspond to a respective modality of the medical imaging data. Continuing with this example, each autoencoder of the at least two autoencoders may be trained on historical medical images of a respective modality of the medical imaging data. Further, the method 800 may include generating, via one or more processors of the ML classifier model, a multi-modal stroke outcome classification of the medical imaging data using the two or more embedding vectors and the contextual information.

Embodiments may also include a time series of medical images for the medical imaging data, wherein the medical imaging data is initial medical imaging data. Further, the method 800 may also include generating, via the one or more autoencoders, one or more additional embedding vectors for one or more additional medical images included in the time series. For example, each embedding vector of the one or more additional embedding vectors may correspond to a respective medical image of the one or more additional medical images. Additionally, the method 800 may include generating, via one or more processors of the ML classifier model, a time series stroke outcome classification of the medical imaging data using the one or more additional embedding vectors, the at least one embedding vector, and the contextual information.

In some embodiments, the method 800 may further include presenting, on a display of a user computing device (e.g., clinician computing device 104), one or more of: the stroke outcome classification, the medical imaging data, or one or more additional classifications for the medical imaging data. In some embodiments, the method 800 includes generating, via one or more processors of a trained ML model, a treatment recommendation for the medical imaging data using one or more of: (i) the at least one embedding vector, (ii) the contextual information, or (iii) the stroke outcome classification.

In the foregoing specification, specific embodiments have been described. However, one of ordinary skill in the art appreciates that various modifications and changes can be made without departing from the scope of the invention as set forth in the claims below. Accordingly, the specification and figures are to be regarded in an illustrative rather than a restrictive sense, and all such modifications are intended to be included within the scope of present teachings. Additionally, the described embodiments/examples/implementations should not be interpreted as mutually exclusive, and should instead be understood as potentially combinable if such combinations are permissive in any way. In other words, any feature disclosed in any of the aforementioned embodiments/examples/implementations may be included in any of the other aforementioned embodiments/examples/implementations.

The benefits, advantages, solutions to problems, and any element(s) that may cause any benefit, advantage, or solution to occur or become more pronounced are not to be construed as a critical, required, or essential features or elements of any or all the claims. The invention is defined solely by the appended claims including any amendments made during the pendency of this application and all equivalents of those claims as issued.

Moreover, in this document, relational terms such as first and second, top and bottom, and the like may be used solely to distinguish one entity or action from another entity or action without necessarily requiring or implying any actual such relationship or order between such entities or actions. The terms "comprises," "comprising," “has”, “having,” “includes”, “including,” “contains”, “containing” or any other variation thereof, are intended to cover a non-exclusive inclusion, such that a process, method, article, or apparatus that comprises, has, includes, contains a list of elements does not include only those elements but may include other elements not expressly listed or inherent to such process, method, article, or apparatus. An element proceeded by “comprises …a”, “has …a”, “includes …a”, “contains …a” does not, without more constraints, preclude the existence of additional identical elements in the process, method, article, or apparatus that comprises, has, includes, contains the element. The terms “a” and “an” are defined as one or more unless explicitly stated otherwise herein. The terms “substantially”, “essentially”, “approximately”, “about” or any other version thereof, are defined as being close to as understood by one of ordinary skill in the art, and in one non-limiting embodiment the term is defined to be within 10%, in another embodiment within 5%, in another embodiment within 1% and in another embodiment within 0.5%. The term “coupled” as used herein is defined as connected, although not necessarily directly and not necessarily mechanically. A device or structure that is “configured” in a certain way is configured in at least that way, but may also be configured in ways that are not listed.

It will be appreciated that some embodiments may be comprised of one or more generic or specialized processors (or “processing devices”) such as microprocessors, digital signal processors, customized processors and field programmable gate arrays (FPGAs) and unique stored program instructions (including both software and firmware) that control the one or more processors to implement, in conjunction with certain non-processor circuits, some, most, or all of the functions of the method and/or apparatus described herein. Alternatively, some or all functions could be implemented by a state machine that has no stored program instructions, or in one or more application specific integrated circuits (ASICs), in which each function or some combinations of certain of the functions are implemented as custom logic. Of course, a combination of the two approaches could be used.

Moreover, an embodiment can be implemented as a computer-readable storage medium having computer readable code stored thereon for programming a computer (e.g., comprising a processor) to perform a method as described and claimed herein. Examples of such computer-readable storage mediums include, but are not limited to, a hard disk, a CD-ROM, an optical storage device, a magnetic storage device, a ROM (Read Only Memory), a PROM (Programmable Read Only Memory), an EPROM (Erasable Programmable Read Only Memory), an EEPROM (Electrically Erasable Programmable Read Only Memory) and a Flash memory. Further, it is expected that one of ordinary skill, notwithstanding possibly significant effort and many design choices motivated by, for example, available time, current technology, and economic considerations, when guided by the concepts and principles disclosed herein will be readily capable of generating such software instructions and programs and ICs with minimal experimentation.

The Abstract of the Disclosure is provided to allow the reader to quickly ascertain the nature of the technical disclosure. It is submitted with the understanding that it will not be used to interpret or limit the scope or meaning of the claims. In addition, in the foregoing Detailed Description, it can be seen that various features are grouped together in various embodiments for the purpose of streamlining the disclosure. This method of disclosure is not to be interpreted as reflecting an intention that the claimed embodiments require more features than are expressly recited in each claim. Rather, as the following claims reflect, inventive subject matter lies in less than all features of a single disclosed embodiment. Thus the following claims are hereby incorporated into the Detailed Description, with each claim standing on its own as a separately claimed subject matter.

Moreover, the patent claims at the end of this patent application are not intended to be construed under 35 U.S.C. § 112(f) unless traditional means-plus-function language is expressly recited, such as “means for” or “step for” language being explicitly recited in the claim(s). The systems and methods described herein are directed to an improvement to computer functionality, and improve the functioning of conventional computers.

What is claimed is:

1. A computer-implemented method for generating a predicted outcome following a cerebrovascular accident, the method comprising:
generating, via one or more autoencoders, at least one embedding vector for medical imaging data corresponding to the cerebrovascular accident,
wherein the at least one embedding vector is an output of an autoencoder trained on historical medical images for a patient population associated with cerebrovascular accidents;
generating, via one or more processors, contextual information for the medical imaging data based on historical electronic health record (EHR) data for the patient population; and
generating, via one or more processors of a machine learning (ML) classifier model, a stroke outcome classification of the medical imaging data using the at least one embedding vector and the contextual information.

2. The computer-implemented method of claim 1, further including:
presenting, on a display of a user computing device, one or more of: the stroke outcome classification, the medical imaging data, or one or more additional classifications for the medical imaging data.

3. The computer-implemented method of claim 1, wherein the historical EHR data includes partially available privileged information for the patient population, and wherein the method further includes:
training, via one or more processors of the ML classifier model, an artificial neural network model to learn a function that maps a standard feature space of historical medical image embeddings and a privileged feature space of the historical EHR data to an outcome.

4. The computer-implemented method of claim 1, further including:
obtaining, via one or more processors of a server computing device, EHR data associated with the medical imaging data,
wherein the stroke outcome classification is generated using the EHR data associated with the medical imaging data.

5. The computer-implemented method of claim 4, wherein the EHR data associated with the medical imaging data includes additional historical medical imaging data, and wherein the method further includes:
generating, via the one or more autoencoders, at least one additional embedding vector for the additional historical medical imaging data,
wherein the stroke outcome classification is generated using the at least one additional embedding vector.

6. The computer-implemented method of claim 1, further including:
training, via one or more processors, the autoencoder on a historical medical imaging dataset to reduce dimensionality of input feature vectors,
wherein imaging data included in the historical medical imaging dataset is mapped to a standardized coordinate system and normalized by applying a brain mask.

7. The computer-implemented method of claim 1, wherein at least one autoencoder of the one or more autoencoders includes a squeeze and excitation network.

8. The computer-implemented method of claim 1, wherein the stroke outcome classification is a length of stay (LOS) classification, a modified Rankin scale (mRS) classification, a recovery timeline classification, or an intervention classification.

9. The computer-implemented method of claim 1, wherein the medical imaging data is magnetic resonance imaging (MRI) data, computed tomography (CT) imaging data, or computed tomography angiography (CTA) imaging data.

10. The computer-implemented method of claim 1, wherein the medical imaging data is multi-modal imaging data, and wherein the method further includes:
generating, via one or more autoencoders, two or more embedding vectors for the medical imaging data using at least two autoencoders of the one or more autoencoders,
wherein each embedding vector of the two or more embedding vectors corresponds to a respective modality of the medical imaging data, and
wherein each autoencoder of the at least two autoencoders is trained on historical medical images of a respective modality of the medical imaging data; and
generating, via one or more processors of the ML classifier model, a multi-modal stroke outcome classification of the medical imaging data using the two or more embedding vectors and the contextual information.

11. The computer-implemented method of claim 1, wherein the medical imaging data includes a time series of medical images, wherein the medical imaging data is initial medical imaging data, and wherein the method further includes:
generating, via one or more autoencoders, one or more additional embedding vectors for one or more additional medical images included in the time series,
wherein each embedding vector of the one or more additional embedding vectors corresponds to a respective medical image of the one or more additional medical images; and
generating, via one or more processors of the ML classifier model, a time series stroke outcome classification of the medical imaging data using the one or more additional embedding vectors, the at least one embedding vector, and the contextual information.

12. The computer-implemented method of claim 1, further including:
generating, via one or more processors of a trained ML model, a treatment recommendation for the medical imaging data using one or more of: (i) the at least one embedding vector, (ii) the contextual information, or (iii) the stroke outcome classification.

13. The computer-implemented method of claim 1, wherein at least one of the one or more autoencoders is trained on historical electronic health record (EHR) data for the patient population.

14. A computing system comprising:
one or more processors; and
one or more memories including computer-executable instructions that, when executed by the one or more processors, cause the computing system to:
generate, via one or more autoencoders, at least one embedding vector for medical imaging data corresponding to a cerebrovascular accident,
wherein the at least one embedding vector is an output of an autoencoder trained on historical medical images for a patient population associated with cerebrovascular accidents;
generate contextual information for the medical imaging data based on historical electronic health record (EHR) data for the patient population; and
generate, via a machine learning (ML) classifier model, a stroke outcome classification of the medical imaging data using the at least one embedding vector and the contextual information.

15. The computing system of claim 14, wherein the historical EHR data includes partially available privileged information for the patient population, and the one or more memories further including computer-executable instructions that, when executed by the one or more processors, cause the computing system to:
train an artificial neural network model to learn a function that maps a standard feature space of historical medical image embeddings and a privileged feature space of the historical EHR data to an outcome.

16. The computing system of claim 14, the one or more memories further including computer-executable instructions that, when executed by the one or more processors, cause the computing system to:
obtain EHR data associated with the medical imaging data,
wherein the stroke outcome classification is generated using the EHR data associated with the medical imaging data.

17. The computing system of claim 14, the one or more memories further including computer-executable instructions that, when executed by the one or more processors, cause the computing system to:
train the autoencoder on a historical medical imaging dataset to reduce dimensionality of input feature vectors,
wherein imaging data included in the historical medical imaging dataset is mapped to a standardized coordinate system and normalized by applying a brain mask.

18. The computing system of claim 14, wherein the medical imaging data is magnetic resonance imaging (MRI) data, computed tomography (CT) imaging data, or computed tomography angiography (CTA) imaging data.

19. The computing system of claim 14, the one or more memories further including computer-executable instructions that, when executed by the one or more processors, cause the computing system to:
generate, via a trained ML model, a treatment recommendation for the medical imaging data using one or more of: (i) the at least one embedding vector, (ii) the contextual information, or (iii) the stroke outcome classification.

20. The computing system of claim 14, the one or more memories further including computer-executable instructions that, when executed by the one or more processors, cause the computing system to:
present, on a display of a user computing device, one or more of: the stroke outcome classification, the medical imaging data, or one or more additional classifications for the medical imaging data.
